(19) United States
(12) Patent Application Publication
(10) Pub. No.: US 2018/0320126 A1
(43) Pub. Date: Nov. 8, 2018

(54) In Vitro Fertilization Fertilized Egg Incubation System

(71) Applicant: Kevin J. Doody, Colleyville, TX (US)

(72) Inventor: Kevin J. Doody, Colleyville, TX (US)

(21) Appl. No.: 15/944,372

(22) Filed: Apr. 3, 2018

Related U.S. Application Data
(60) Provisional application No. 62/501,375, filed on May 4, 2017.

(51) Int. Cl. C12M 1/00; A61B 17/435; A61B 17/43; C12N 5/073; C12N 5/071; C12N 5/076; C12M 3/00

(52) U.S. Cl. CPC C12M 41/14; A61B 17/435; A61B 17/43; C12N 2523/00; C12N 5/0608; C12N 5/061; C12M 21/06; C12N 5/0604

(57) ABSTRACT

The subject matter of this specification can be embodied in, among other things, a method for controlling an incubator that includes receiving by a controller a temperature profile, and varying by the controller a temperature of an incubator based on the temperature profile.

### CLAIM OF PRIORITY

This application claims priority to U.S. Provisional Application Ser. No. 62/501,375, filed on May 4, 2017, the entire contents of which are hereby incorporated by reference.

### TECHNICAL FIELD

This disclosure relates to incubation systems used, for example, in in vitro fertilization (IVF) processes.

### BACKGROUND

In vitro fertilization (IVF) is a medical procedure in which an egg is fertilized by sperm outside of the body. IVF generally involves several steps: ovulation induction, egg retrieval, sperm retrieval, fertilization, embryo culture and embryo transfer. In order to keep fertilized eggs viable, the eggs are placed in a nutritive liquid (culture medium) and are kept in an incubating environment that is conducive to the survival and growth of the fertilized eggs (e.g., developing embryos).

In the case of mammals (e.g., humans), that incubating environment is similar to the female reproductive tract (e.g., fallopian tubes, uterus) with regard to temperature, gas concentrations, etc. Some existing IVF incubation techniques involve placing the inseminated eggs in a container (e.g., sample tube), and temporarily inserting the container into the female's body (e.g., the prospective mother's vagina).

Other existing IVF incubation techniques involve placing the inseminated eggs and developing embryos in an environmentally-controlled mechanical incubator. In the case of human IVF, the temperature of the incubator is held at the average human internal body temperature of about 37° C. (98.6° F.). Such incubation systems typically have sensors and temperature controls to maintain that set temperature throughout the incubation process, although in some cases the temperature may vary somewhat given a design and temperature control process for a given incubation system (e.g., hysteresis in the temperature control loop). That said, the design of the temperature control in such systems is to maintain a set temperature throughout the incubation process.

### SUMMARY

In general, this document describes incubation systems used, for example, in in vitro fertilization (IVF) processes.

In a first aspect, a method for controlling an incubator includes receiving by a controller a temperature profile, and varying by the controller a temperature of an incubator based on the temperature profile.

Various implementations can include some, all, or none of the following features. The temperature profile can include a first description of a first temperature state having a first temperature to be maintained for a first period of time, and a second description of second temperature state having a second temperature, different from the first temperature, to be maintained for a second period of time that is disjoint from the first period of time. The temperature profile can be descriptive of a mammalian female's diurnal internal body temperature cycle. The temperature profile can be descriptive of a temperature cycle that varies from less than or equal to 39° C. to greater than or equal to 35° C. over a period of greater than 15 hours to less than 48 hours. Varying a temperature of an incubator based on the temperature profile can include operating a heater configured to heat the incubator to the first temperature, operating the heater to maintain the incubator at the first temperature for the first period of time, operating, after the first period of time has elapsed, the heater to heat the incubator at the second temperature, and operating the heater to maintain the incubator at the second temperature for the second period of time. The method can include operating, after the second period of time has elapsed, the heater to heat the incubator to the first temperature, and operating the heater to maintain the incubator at the first temperature for the first period of time. The method can also include receiving a temperature change rate threshold value, and operating the heater to change the temperature of the incubator from one of the first temperature and the second temperature to the other of the first temperature and the second temperature at a temperature change rate based on the change rate threshold value.

In a second aspect, a method for incubating includes receiving an incubation sample, placing the incubation sample in an incubator comprising a heater, identifying a temperature profile, controlling the heater to heat the incubator to a first temperature based on the temperature profile, controlling the heater to incubate the incubation sample at the first temperature for a first period of time based on the temperature profile, controlling, after the first period of time has elapsed, the heater to heat the incubator to about a second temperature, different from the first temperature, based on the temperature profile, and controlling the heater to incubate the incubation sample at the second temperature for a second period of time based on the temperature profile.

Various implementations can include some, all, or none of the following features. The temperature profile can be descriptive of a mammalian female diurnal internal body temperature cycle. The temperature profile can be descriptive of a temperature cycle that varies from less than or equal to 39° C. to greater than or equal to 35° C. over a period of greater than 15 hours to less than 48 hours. The incubation sample includes at least one of an embryo, a fertilized egg, an unfertilized egg, and sperm.

In a third aspect, an incubation system includes a sample holder, a heater configured to heat the sample holder, a temperature sensor configured to provide a temperature feedback signal, and a controller configured to receive a temperature profile, and vary a temperature of the heater based on the temperature profile and the temperature feedback signal.

Various embodiments can include some, all, or none of the following features. The sample holder can hold at least one of an embryo, a fertilized egg, an unfertilized egg, and sperm. The temperature profile can include a first description of a first temperature state having a first temperature to be maintained for a first period of time, and a second description of second temperature state having a second temperature, different from the first temperature, to be maintained for a second period of time that is disjoint from the first period of time. The temperature profile can be descriptive of a mammalian female's diurnal internal body temperature cycle. The temperature profile can be descriptive of a temperature cycle that varies from less than or equal to 39° C. to greater than or equal to 35° C. over a period of greater than 15 hours to less than 48 hours. Varying a temperature of the heater can be based on the temperature profile and the temperature feedback signal can include operating the heater to heat the sample holder to the first temperature, operating the heater to maintain the sample holder at the first temperature for the first period of time, operating, after the first period of time has elapsed, the heater to heat the sample holder at the second temperature, and operating the heater to maintain the sample holder at the second temperature for the second period of time. Varying a temperature of the heater based on the temperature profile and the temperature feedback signal can include operating, after the second period of time has elapsed, the heater to heat the sample holder to the first temperature, and operating the heater to maintain the sample holder at the first temperature for the first period of time. The controller can be further configured to receive a temperature change rate threshold value, and operate the heater to change the temperature of the sample holder from one of the first temperature and the second temperature to the other of the first temperature and the second temperature at a temperature change rate that is based on the change rate threshold value.

The systems and techniques described here may provide one or more of the following advantages. First, a system can provide a more physiologic environmental temperature for inseminated eggs and/or developing embryos. Second, the system can provide a controlled, variable environmental temperature that promotes the viability of inseminated eggs and/or developing embryos. Third, the system can automate and emulate the natural cyclical variance of physiologic environmental temperature of a normal, reproductively heathy female's reproductive tract.

The details of one or more implementations are set forth in the accompanying drawings and the description below. Other features and advantages will be apparent from the description and drawings, and from the claims.

### DESCRIPTION OF DRAWINGS

FIG. 1 is a schematic diagram that shows an example incubation system.

FIG. 2 is a chart of an example human diurnal body temperature cycle.

FIG. 3 shows an example user interface for an incubation system.

FIG. 4 is flow chart that shows an example of an incubation process.

FIG. 5 is a schematic diagram of an example of a generic computer system.

### DETAILED DESCRIPTION

This document describes systems and techniques for the incubation of eggs that have been fertilized through an in vitro fertilization (IVF) processes. In general, the systems and techniques described in this document differ from existing IVF incubators by including a temperature control unit that intentionally effects a programmed variance in temperature during the incubation process, as opposed to a temperature control unit that holds temperature to a set temperature (e.g., 37° C. for humans).

In at least the case of humans, core body temperature is not always a constant 37° C. Instead, core temperatures vary slightly in a diurnal (e.g., daily) cycle. The IVF incubator can be programmed to vary incubation temperatures in ways that emulate the natural, diurnal, temperature variance of a typical or particular female's reproductive tract.

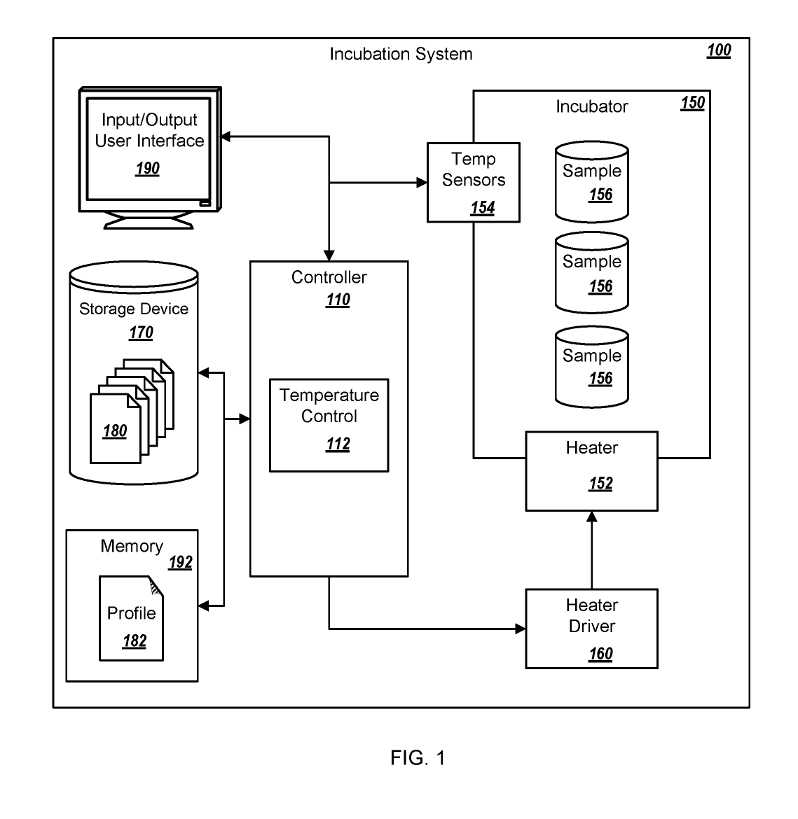

FIG. 1 is a schematic diagram that shows an example incubation system 100. The system 100 includes a controller 110 configured to control the temperature and other operational parameters of an incubator 150. The incubator 150 includes a heater 152, driven by a heater driver circuit 160, and one or more temperature sensors 154 as part of an apparatus that is configured to control the temperature of one or more sample containers 156. In some implementations, the sample containers 156 can hold biological samples, such eggs, sperm, fertilized eggs, or live embryos. In some examples, the fertilized eggs and/or embryos can be the result of an IVF process, and can be heated by the incubator 150 as they await implantation within a candidate female (e.g., prospective mother). In some embodiments, the incubator 150 can be configured to control other environmental conditions of the samples containers 156, such as humidity, ambient gas concentrations, pressure, light levels, and/or other appropriate factors.

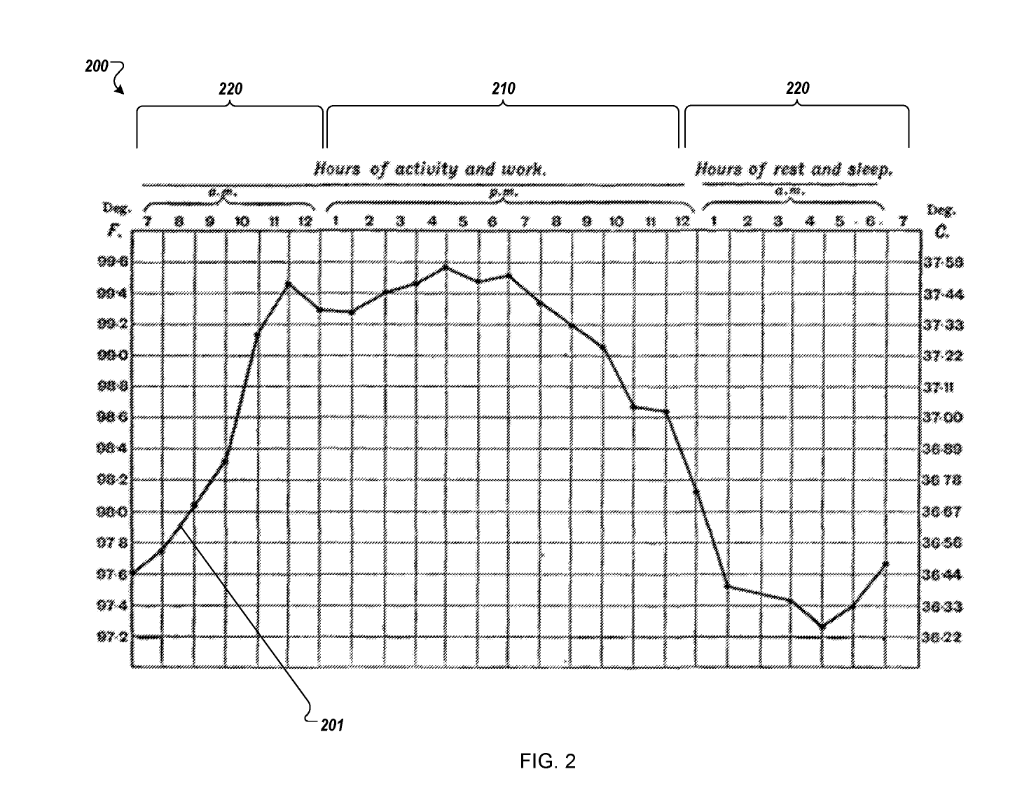

A storage device 170 (e.g., a hard drive) in data communications with the controller 110 stores a collection 180 of temperature profiles. Each of the temperature profiles in the collection 180 can represent a one or more temperature setpoints and one or more corresponding time periods. In a simple example, a profile can describe a first temperature setting (e.g., 37.5° C.) that should be held for a first period of time (e.g., 6 am to 9 pm) and a second temperature (e.g., 36.8° C.) that should be held or a second period of time (e.g., from 9 pm to 6 am). In another example, a profile can describe the diurnal temperature cycle within the reproductive tract of an individual female. An example of a temperature profile is discussed further in the description of FIG. 2.

The controller 110 receives input from a user (e.g., doctor, clinician, embryologist) through a user interface 190 (e.g., display, audio, keyboard, mouse) to select a profile 182 to be retrieved from the collection 180 and be held in a memory 192 (e.g., random access memory of the controller 110) for use by the controller 110.

The controller 110 includes a temperature control module 112. The temperature control module 112 is configured to control the heater driver circuit 160 based on feedback signals from the temperature sensors 154 and a temperature setpoint. The temperature setpoint is varied based on the times and temperatures described by temperature profile 182.

FIG. 2 is a chart 200 of an example human diurnal body temperature cycle 201. In some implementations, the example collection 180 (of FIG. 1) can include temperature profiles (e.g., the example temperature profile 182) that describe the cycle 201.

In general, humans' internal body temperatures are not constant. Instead, as illustrated by the chart 200, human body temperature varies on a periodic cycle. In the example cycle 201, the subject's internal body temperature varies from a high temperature of about 37.56° C. to a low temperature of about 36.22° C. over a 24-hour period. In the illustrated example, the temperature is higher during a time period 210 (e.g., daytime hours when the subject is awake and active) and the temperature is lower during a time period 220 (e.g., nighttime hours when the subject is at rest and asleep).

The cycle 201 is just one example. In some examples, the times, durations, and or quantities of time periods may be different from the time periods 210, 220, depending on the subject. In some examples, the high temperatures, the low temperatures, the rates of temperature changes between time periods, and the ranges of temperature variability within individual time periods may be different depending on the subject. In some examples, the cycle 201 may occur over a period other than 24 hours. In some examples, the durations of the time periods 210, 220 can be determined randomly within predetermined time limits (e.g., a random number generator can be used in a process of picking a duration of the time period 210 that is between one hour and sixteen hours). In some examples, the high temperatures and/or the low temperatures may be determined randomly within predetermined temperature limits (e.g., a random number generator can be used in a process of picking a temperature setpoint that is between 36° C. and 38° C.).

In some embodiments, the cycle 201 can represent the diurnal body temperature cycle of an individual female's reproductive tract. For example, the cycle 201 may be measured or otherwise determined for a prospective mother for use (e.g., by the example system 100 of FIG. 1) in incubating IVF-fertilized eggs prior to implantation. In some examples, the cycle 201 can represent the diurnal body temperature cycle of a representative females' reproductive tract. For example, the cycle 201 may be measured or otherwise determined from a healthy female for use in incubating IVF-fertilized eggs for implantation in another female (e.g., who may have an atypical temperature cycle that is less conducive for successful incubation) prior to implantation. In some embodiments, the cycle 201 can represent a diurnal body temperature cycle of a population of females' reproductive tracts. For example, the diurnal temperature cycles of multiple females may be determined, and the multiple cycles may be mathematically combined (e.g., averaged on an hour-by-hour or minute-by-minute basis) to determine a representative diurnal temperature cycle for use in incubating the IVF-fertilized eggs of multiple prospective mothers at the same time, prior to implantation.

Temperature profiles, such as the example temperature profile 182, can describe a programmed (e.g., intentional) temperature variance that emulates the natural cyclical temperature variance of female core body temperatures and/or reproductive tract temperatures. In some embodiments, programmed temperature variance options may one of the following: diurnal variance corresponding generally to daily body temperature patterns of a mother (e.g., as illustrated by the chart 200), a periodic programmed variance with a cycle of less than or greater than 24 hours, a periodic programmed variance that mimics a typical variation in temperature of a female reproductive tract (e.g., the fallopian tube), a user-defined temperature variance profile designed to be optimal for a specific patient into which the incubated embryo will be implanted, and any other temperature profiles determined to increase the likelihood of producing viable embryos for implantation.

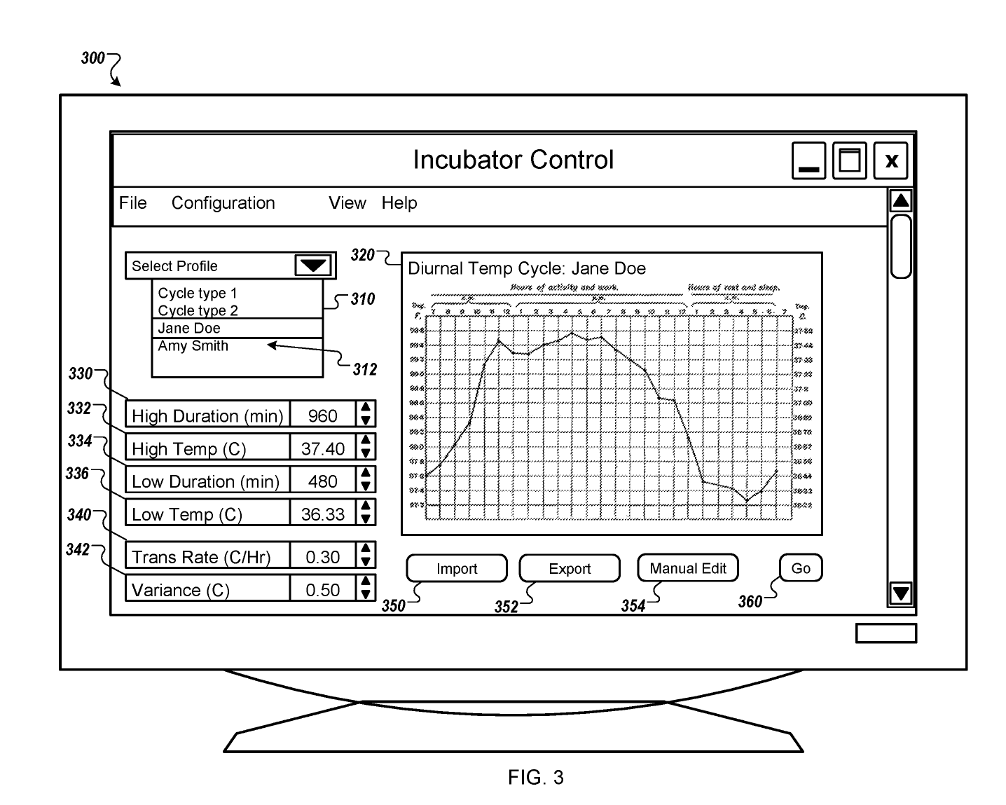

FIG. 3 shows an example user interface 300 for an incubation system. In general, the user interface 300 provides at least a portion of the inputs and outputs with which a user can interact to control an incubation system such as the example incubation system 100 of FIG. 1. In some embodiments, the user interface 300 can be all or part of the example user interface 190.

The user interface 190 includes a profile selector 310. The profile selector 310 provides an interface for the selection of one or more of a collection of temperature profiles 312. In some implementations, the collection 312 can be the example collection 180. In the illustrated example, a temperature profile for the subject “Jane Doe” has been selected. In some implementations, the selected temperature profile can be the temperature profile 182. Once selected, the temperature profile for “Jane Doe” is displayed to the user as a chart component 320. In some embodiments, the chart component 320 can be configured for input as well as display output. For example, the user can use a mouse, touchscreen, or other input device to modify or otherwise edit the times and/or temperatures of the points that make up the temperature cycle presented by the chart component 320.

A high temperature duration component 330 provides the user with a way to view and/or modify a duration of time described by a selected temperature profile, for example to describe the length of time during which a high portion of a diurnal temperature cycle is to be emulated within the example incubator 150. A high temperature setpoint component 332 provides the user with a way to view and/or modify the temperature that is to be provided during a high portion of a diurnal temperature cycle, for example, by the example incubator 150. In the illustrated example, the high temperature period is set to last for 960 minutes at a temperature of 37.40° C.

A low temperature duration component 334 provides the user with a way to view and/or modify a duration of time described by a selected temperature profile, for example to describe the length of time during which a low portion of a diurnal temperature cycle is to be emulated within the example incubator 150. A low temperature setpoint component 336 provides the user with a way to view and/or modify the temperature that is to be provided during a low portion of a diurnal temperature cycle, for example, by the example incubator 150. In the illustrated example, the low temperature period is set to follow the high temperature period, and last for 480 minutes at a temperature of 36.33° C. (e.g., before restarting the cycle with another high temperature period).

A transition rate component 340 provides the user with a way to view and/or modify a target or maximum rate of temperature change between two temperatures. For example, the transition rate component 340 can be set to “0.3° C./hr” to configure the incubation system 100 to control the heater 152 to change the temperature within the incubator 150 from the temperature indicated by the low temperature setpoint component 336 to the temperature indicated by the high temperature setpoint 332 at a rate that is equal to or less than the value indicated by the transition rate component 340 (e.g., to change from 36.33° C. to 37.40° C. at no more than 0.3° C./hr in the illustrated example).

A variance component 342 provides the user with a way to view and/or modify an amount by which a temperature setpoint may vary. In some implementations, an incubation process may be further enhanced by allowing or purposely causing a temperature to vary by a predetermined amount about a selected temperature setpoint (e.g., to stimulate embryonic development by emulating smaller temperature swings that might occur within a mother's body during various daily activities). In the illustrated example, the incubator 150 can be heated to a target high temperature of 37.40° C., and vary about that temperature by 0.5° C. while changing at no more than 0.3° C. per hour.

An import button 350, when pressed, can cause the user interface 300 to present controls with which a user can interact to import a temperature profile. For example, the user can press the import button 350 to load a temperature profile from a portable media device or network location into the example storage device 170 or the example memory 192. In some implementations, a conversion process can be implemented, for example, to import temperature profiles stored in a format that is not native to the controller 110 and transform them for use by the controller 110.

An export button 352, when pressed, can cause the user interface 300 to present controls with which a user can interact to export a temperature profile. For example, the user can press the export button 352 to save a temperature profile from the example storage device 170 or the example memory 192 to a portable media device or network location. In some implementations, a conversion process can be implemented, for example, to transform temperature profiles from a format used by the controller 110 and store them in a format that may not be native to the controller 110 (e.g., text, CSV, XML).

An activation button 360 can be pressed to cause the selected temperature profile to be used in an incubation process. For example, the activation button 360 can be pressed to cause the example controller 110 to programmatically vary the temperature of the incubator 150 over time based on the selected temperature profile and/or the settings indicated by the components 330-342.

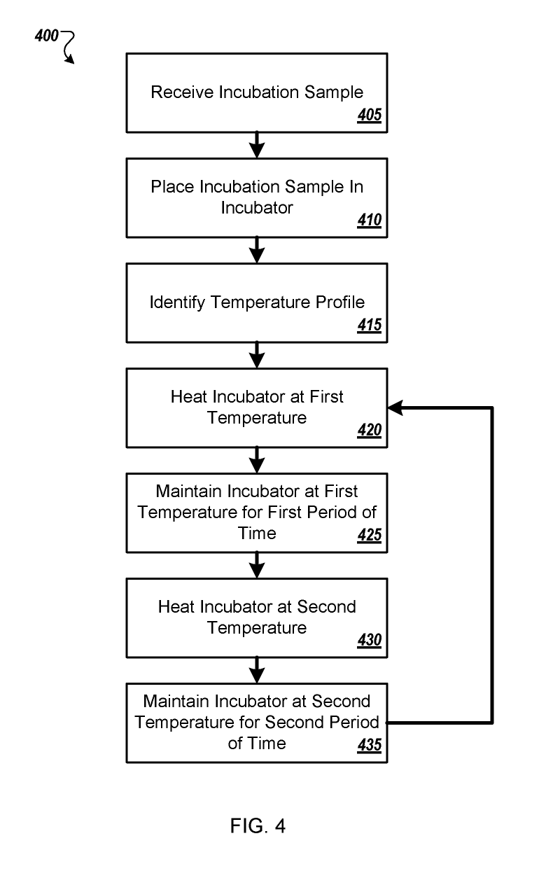

FIG. 4 is flow chart that shows an example of an incubation process 400. In some implementations, the process 400 can be performed by the example incubation system 100 of FIG. 1.

At 405, an incubation sample is received. For example, fertilized egg can be placed in one of the example sample containers 156, or sperm can be placed in one of the sample containers 156 along with an unfertilized egg for IVF fertilization within the sample container 156. In some implementations, the incubation sample can include at least one of an embryo, a fertilized egg, an unfertilized egg, and sperm.

At 410 the incubation sample is placed in an incubator that has a heater. For example, the sample containers 156 can be placed in the example incubator 150, which includes the example heater 152 configured to heat the incubator 150 such that the sample containers 156 and their contents (e.g., fertilized eggs, embryos) are heated.

At 415, a temperature profile is identified. For example, a user can interact with the example user interface 300 of FIG. 3 to select the temperature profile 182 from the collection temperature profiles 180 and load it from the storage device 170 into memory 192 for use by the controller 110.

In some implementations, the temperature profile can be descriptive of a mammalian female diurnal internal body temperature cycle. For example, the temperature profile can describe the repeating, daily temperature changes within a human female's reproductive tract. In some implementations, the temperature profile can be descriptive of a temperature cycle that varies from about 39° C. to about 35° C. (e.g., 39° C.+/−2° C.). In some implementations, the temperature profile can be descriptive of a temperature cycle that varies from about 37.5° C. to about 36.5° C. (e.g., 39° C.+/−0.5° C.). In some implementation, the temperature profile can describe temperature variations that occur over a period of about 20 hours to about 48 hours (e.g., a 24-hour daily wake/sleep cycle). For example, the example temperature profile 201 of FIG. 2 describes a temperature cycle that varies from a high temperature of about 37.56° C. during typical waking hours to a low temperature of about 36.22° C. during typical sleeping hours.

At 420 the heater is controlled to heat the incubator to a first temperature based on the temperature profile. For example, the temperature profile 182 can indicate that the heater 152 is to be used to heat the incubator 150 (and the sample containers 156) to a temperature that is slightly above the average human core body temperature of 37° C.

At 425, the heater is controlled to incubate the incubation sample at the first temperature for a first period of time based on the temperature profile. For example, the temperature profile 182 can indicate that the heater 152 is to be used to maintain the incubator 150 (and the sample containers 156) at about slightly more than 37° C. for sixteen hours (e.g., approximately the amount of time an average person is awake and active).

At 430, after the first period of time has elapsed, the heater is controlled to heat the incubator to about a second temperature, different from the first temperature, based on the temperature profile. For example, the temperature profile 182 can indicate that after the sixteen-hour period has passed, the heater 152 is to be used to heat the incubator 150 (and the sample containers 156) to a temperature that is slightly below the average human core body temperature of 37° C.

At 435, the heater is controlled to incubate the incubation sample at the second temperature for a second period of time based on the temperature profile. For example, the temperature profile 182 can indicate that the heater 152 is to be used to maintain the incubator 150 (and the sample containers 156) at about slightly less than 37° C. for eight hours (e.g., approximately the amount of time an average person is resting or asleep).

In some implementations, when the second period of time expires the heater can be controlled to heat the incubator at the first temperature for another one of the first periods of time. For example, the process 400 may repeat by performing step 420 again, forming an operational loop that emulates the repeating diurnal temperature cycle of a female reproductive tract.

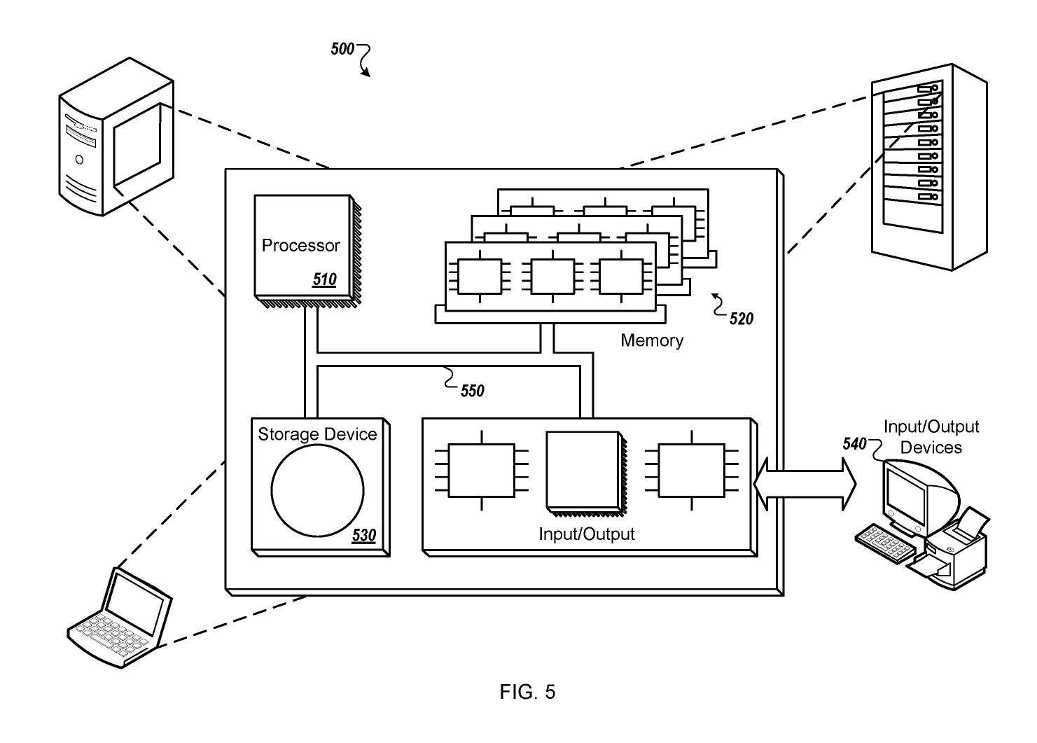

FIG. 5 is a schematic diagram of an example of a generic computer system 500. The system 500 can be used for the operations described in association with the method 400 according to one implementation. For example, the system 500 may be included in either or both of the example controller 110 and the temperature control module 112.

The system 500 includes a processor 510, a memory 520, a storage device 530, and an input/output device 540. Each of the components 510, 520, 530, and 540 are interconnected using a system bus 550. The processor 510 is capable of processing instructions for execution within the system 500. In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the system 500. In one implementation, the memory 520 is a computer-readable medium. In one implementation, the memory 520 is a volatile memory unit. In another implementation, the memory 520 is a non-volatile memory unit.

The storage device 530 is capable of providing mass storage for the system 500. In one implementation, the storage device 530 is a computer-readable medium. In various different implementations, the storage device 530 may be a floppy disk device, a hard disk device, an optical disk device, or a tape device.

The input/output device 540 provides input/output operations for the system 500. In one implementation, the input/output device 540 includes a keyboard and/or pointing device. In another implementation, the input/output device 540 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

Although a few implementations have been described in detail above, other modifications are possible. In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

What is claimed is:

1. A method for controlling an incubator, comprising:
receiving, by a controller, a temperature profile; and
varying, by the controller, a temperature of an incubator based on the temperature profile.

2. The method of claim 1, wherein the temperature profile comprises:
a first description of a first temperature state having a first temperature to be maintained for a first period of time; and
a second description of second temperature state having a second temperature, different from the first temperature, to be maintained for a second period of time that is disjoint from the first period of time.

3. The method of claim 2, wherein the temperature profile is descriptive of a mammalian female's diurnal internal body temperature cycle.

4. The method of claim 2, wherein the temperature profile is descriptive of a temperature cycle that varies from less than or equal to 39° C. to greater than or equal to 35° C. over a period of greater than 15 hours to less than 48 hours.

5. The method of claim 2, wherein varying a temperature of an incubator based on the temperature profile comprises:
operating a heater configured to heat the incubator to the first temperature;
operating the heater to maintain the incubator at the first temperature for the first period of time;
operating, after the first period of time has elapsed, the heater to heat the incubator at the second temperature; and
operating the heater to maintain the incubator at the second temperature for the second period of time.

6. The method of claim 5, further comprising:
operating, after the second period of time has elapsed, the heater to heat the incubator to the first temperature; and
operating the heater to maintain the incubator at the first temperature for the first period of time.

7. The method of claim 2, further comprising:
receiving a temperature change rate threshold value; and
operating the heater to change the temperature of the incubator from one of the first temperature and the second temperature to the other of the first temperature and the second temperature at a temperature change rate based on the change rate threshold value.

8. A method for incubating, comprising:
receiving an incubation sample;
placing the incubation sample in an incubator comprising a heater;
identifying a temperature profile;
controlling the heater to heat the incubator to a first temperature based on the temperature profile;
controlling the heater to incubate the incubation sample at the first temperature for a first period of time based on the temperature profile;
controlling, after the first period of time has elapsed, the heater to heat the incubator to about a second temperature, different from the first temperature, based on the temperature profile; and
controlling the heater to incubate the incubation sample at the second temperature for a second period of time based on the temperature profile.

9. The method of claim 8, wherein the temperature profile is descriptive of a mammalian female diurnal internal body temperature cycle.

10. The method of claim 8, wherein the temperature profile is descriptive of a temperature cycle that varies from less than or equal to 39° C. to greater than or equal to 35° C. over a period of greater than 15 hours to less than 48 hours.

11. The method of claim 8, wherein the incubation sample comprises at least one of an embryo, a fertilized egg, an unfertilized egg, and sperm.

12. An incubation system comprising:
a sample holder;
a heater configured to heat the sample holder;
a temperature sensor configured to provide a temperature feedback signal; and
a controller configured to:
receive a temperature profile; and
vary a temperature of the heater based on the temperature profile and the temperature feedback signal.

13. The incubation system of claim 12, wherein the sample holder holds at least one of an embryo, a fertilized egg, an unfertilized egg, and sperm.

14. The incubation system of claim 12, wherein the temperature profile comprises:
a first description of a first temperature state having a first temperature to be maintained for a first period of time; and
a second description of second temperature state having a second temperature, different from the first temperature, to be maintained for a second period of time that is disjoint from the first period of time.

15. The incubation system of claim 14, wherein the temperature profile is descriptive of a mammalian female's diurnal internal body temperature cycle.

16. The incubation system of claim 14, wherein the temperature profile is descriptive of a temperature cycle that varies from less than or equal to 39° C. to greater than or equal to 35° C. over a period of greater than 15 hours to less than 48 hours.

17. The incubation system of claim 14, wherein varying a temperature of the heater based on the temperature profile and the temperature feedback signal comprises:
operating the heater to heat the sample holder to the first temperature;
operating the heater to maintain the sample holder at the first temperature for the first period of time;
operating, after the first period of time has elapsed, the heater to heat the sample holder at the second temperature; and
operating the heater to maintain the sample holder at the second temperature for the second period of time.

18. The incubation system of claim 17, wherein varying a temperature of the heater based on the temperature profile and the temperature feedback signal comprises:
operating, after the second period of time has elapsed, the heater to heat the sample holder to the first temperature; and
operating the heater to maintain the sample holder at the first temperature for the first period of time.

19. The incubation system of claim 14, wherein the controller is further configured to:
receive a temperature change rate threshold value; and
operate the heater to change the temperature of the sample holder from one of the first temperature and the second temperature to the other of the first temperature and the second temperature at a temperature change rate that is based on the change rate threshold value.
